(19) United States
(12) Patent Application Publication
(10) Pub. No.: US 2020/0190595 A1
(43) Pub. Date: Jun. 18, 2020

(54) KIT FOR EVALUATING GENE MUTATION RELATED TO MYELOPROLIFERATIVE TUMOR

(71) Applicants: Toyo Kohan Co., Ltd., Shinagawa-ku, Tokyo (JP); Yamaguchi University, Yamaguchi-shi, Yamaguchi (JP)

(72) Inventors: Emi Takamitsu, Kudamatsu-shi, Yamaguchi (JP); Mitsuyoshi Oba, Kudamatsu-shi, Yamaguchi (JP); Junichi Morihiro, Kudamatsu-shi, Yamaguchi (JP); Hirofumi Yamano, Kudamatsu-shi, Yamaguchi (JP); Toshiaki Yujiri, Ube-shi, Yamaguchi (JP); Masafumi Matsuguma, Ube-shi, Yamaguchi (JP)

(73) Assignees: Toyo Kohan Co., Ltd., Shinagawa-ku, Tokyo (JP); Yamaguchi University, Yamaguchi-shi, Yamaguchi (JP)

(21) Appl. No.: 16/625,877

(22) PCT Filed: Jun. 28, 2018
(86) PCT No.: PCT/JP2018/024510

§ 371 Date: Dec. 23, 2019

(30) Foreign Application Priority Data
Jun. 28, 2017 (JP) 2017-125903

(51) Int. Cl. C12Q 1/6886; C12Q 1/6827; C12N 15/113

(52) U.S. Cl. CPC C12Q 1/6886; C12Q 2600/156; C12N 15/113; C12Q 1/6827

(57) ABSTRACT

The present invention easily identifies a genotype for gene mutations related to myeloproliferative neoplasms. The present invention comprises a mutant probe that specifically hybridizes with a gene mutation related to myeloproliferative neoplasms in JAK2, a mutant probe that specifically hybridizes with a gene mutation related to myeloproliferative neoplasms in CALR, and a mutant probe that specifically hybridizes with a gene mutation related to myeloproliferative neoplasms in MPL.

### TECHNICAL FIELD

The present invention relates to a probe set that can evaluate gene mutations useful as diagnostic items for myeloproliferative neoplasms, and a microarray having the probe set.

### BACKGROUND ART

Myeloproliferative neoplasms (MPN) are diseases that occurs due to the oncogenesis of myeloid cells. MPN is characterized by marked proliferation of myeloid cells (such as granulocytes, blasts, bone marrow megakaryocytes, and mast cells). MPN include chronic myelogenous leukemia (CML), chronic neutrophilic leukemia (CNL), polycythemia vera (PV), primary myelofibrosis (PMF), essential thrombocythemia (ET), chronic eosinophilic leukemia (CEL), hypereosinophilic syndrome (HES), mastocytosis and myeloproliferative neoplasms, unclassifiable (MPN, U).

As described in Non Patent Literature 1, the diagnosis of MPN uses clinical parameters, bone marrow morphology, and gene mutation data as indices. For patients with Philadelphia chromosome negative, MPN except CML can be diagnosed by diagnosing with these in combination. As the gene mutation data, mutation information, specifically on three genes: JAK2, CALR and MPL, and additionally on ASXL1, EZH2, TET2, IDH1/IDH2, SRSF2 and SF3B1 are employed. In particular, since JAK2, CALR, and MPL are considered to be the molecular basis of the onset of MPN, the presence or absence of mutations in these genes is an important factor in the definitive diagnosis of MPN.

Furthermore, Non Patent Literature 2 discloses, with respect to JAK2, that JAK2V617F mutation (substitution mutation at position 617 of valine to phenylalanine) is frequently observed in PV, ET and PMF, and that, in addition to the above mutation, an insertion/deletion mutation in exon 12 is observed in a small number of PV. JAK2 (Janus activating kinase 2) is a gene encoding a protein that controls signals of an erythropoietin receptor.

Non Patent Literature 2 further discloses, with respect to MPL, that MPLW515L/K mutant PMF was found in PMF and ET. MPL is a gene encoding a thrombopoietin receptor.

Non Patent Literature 2 further discloses, with respect to CALR, that type 1 mutation of 52-base deletion and type 2 mutation of 5-base insertion are the most frequent, and these mutations are observed in ET and PMF. It is disclosed that type 1 mutations are more frequent in PMF, and associated with conversion to myelofibrosis in ET. CALR is a gene encoding calreticulin that is one of the molecular chaperones of vesicles.

Furthermore, Patent Literature 1 discloses a fluorescently labeled probe that is specific to JAK2V617F site, as a method for analyzing mutations in JAK2 gene. Patent Literature 2 discloses a technique for detecting a mutation different from the JAK2V617F mutation. The mutation was found in a patient who is negative for the JAK2V617F mutation but has myeloproliferative neoplasms.

Furthermore, Patent Literature 3 discloses probe sets for detecting W515K and W515L mutations in MPL, as probes for detecting an MPL gene polymorphism.

Furthermore, Patent Literature 4 discloses a technique for identifying mutations in CALR.

### CITATION LIST

### Patent Literature

Patent Literature 1: JP Patent Publication (Kokai) No. 2012-034580 A
Patent Literature 2: WO 2009/060804
Patent Literature 3: WO 2011/052755
Patent Literature 4: JP Patent Publication (Kohyo) No. 2016-537012 A

### Non Patent Literature

Non Patent Literature 1: Francesco Passamonti and Margherita Maffioli, Hematology 2016, p. 534-542
Non Patent Literature 2: NCCN Clinical Practice Guidelines in Oncology (NCCN Guidelines), Myeloproliferative Neoplasms, Version 2. 2017, Oct. 19, 2016

### SUMMARY OF INVENTION

### Technical Problem

However, in the prior art, there was no means to easily detect the presence or absence of mutations for gene mutations related to myeloproliferative neoplasms, and there has been a problem in diagnoses of myeloproliferative neoplasms that information on the gene mutations described above to be diagnosed cannot be easily used.

Accordingly, considering such circumstances, an object of the present invention is to provide a kit for evaluating a gene mutation that can easily determine the presence or absence of gene mutations for gene mutations related to myeloproliferative neoplasms.

### Solution to Problem

The present invention includes the following.

(1) A kit for evaluating a gene mutation related to myeloproliferative neoplasms, comprising: a mutant probe that specifically hybridizes with a gene mutation related to myeloproliferative neoplasms in JAK2, a mutant probe that specifically hybridizes with a gene mutation related to myeloproliferative neoplasms in CALR, and a mutant probe that specifically hybridizes with a gene mutation related to myeloproliferative neoplasms in MPL.

(2) The kit for evaluating a gene mutation according to (1), wherein the gene mutation related to myeloproliferative neoplasms in JAK2 is a V617F mutation.

(3) The kit for evaluating a gene mutation according to (1), wherein the gene mutation related to myeloproliferative neoplasms in CALR is a type 1 mutation of 52-base deletion in which 52 bases from positions 513 to 564 are deleted in a nucleotide sequence represented by SEQ ID NO: 2 and/or a type 2 mutation of 5-base insertion in which TTGTC is inserted between positions 568 and 569 in the nucleotide sequence represented by SEQ ID NO: 2.

(4) The kit for evaluating a gene mutation according to (1), wherein the gene mutation related to myeloproliferative neoplasms in MPL is a W515K mutation and/or W515L mutation.

(5) The kit for evaluating a gene mutation according to (1), comprising a common probe that hybridizes with a region excluding the gene mutation related to myeloproliferative neoplasms in CALR.

(6) The kit for evaluating a gene mutation according to (1), wherein the mutant probe that specifically hybridizes with a gene mutation related to myeloproliferative neoplasms in JAK2 is an oligonucleotide comprising CTCCACAGAAACATACTCC (SEQ ID NO: 4).

(7) The kit for evaluating a gene mutation according to (1), wherein the gene mutation related to myeloproliferative neoplasms in CALR is a type 1 mutation of 52-base deletion in which 52 bases from positions 513 to 564 are deleted in a nucleotide sequence represented by SEQ ID NO: 2, and the mutant probe that specifically hybridizes with the gene mutation related to myeloproliferative neoplasms in CALR is an oligonucleotide comprising TCCTTGTCCTCTGCTCC (SEQ ID NO: 5).

(8) The kit for evaluating a gene mutation according to (1), wherein the gene mutation related to myeloproliferative neoplasms in CALR is a type 2 mutation of 5-base insertion in which TTGTC is inserted between positions 568 and 569 in the nucleotide sequence represented by SEQ ID NO: 2, and the mutant probe that specifically hybridizes with the gene mutation related to myeloproliferative neoplasms in CALR is an oligonucleotide comprising ATCCTCCGACAATTGTCCT (SEQ ID NO: 6).

(9) The kit for evaluating a gene mutation according to (1), wherein the gene mutation related to myeloproliferative neoplasms in MPL is a W515K mutation, and the mutant probe that specifically hybridizes with the gene mutation related to myeloproliferative neoplasms in MPL is an oligonucleotide comprising GAAACTGCTTCCTCAGCA (SEQ ID NO: 7).

(10) The kit for evaluating a gene mutation according to (1), wherein the gene mutation related to myeloproliferative neoplasms in MPL is a W515L mutation, and the mutant probe that specifically hybridizes with the gene mutation related to myeloproliferative neoplasms in MPL is an oligonucleotide comprising GGAAACTGCAACCTCAG (SEQ ID NO: 8).

(11) The kit for evaluating a gene mutation according to (5), wherein the common probe is an oligonucleotide comprising a sequence of positions 397 to 659 in a nucleotide sequence of CALR gene represented by SEQ ID NO: 2.

(12) The kit for evaluating a gene mutation according to (5), wherein the common probe is an oligonucleotide comprising CTCCTCATCCTCATCTTTGTC (SEQ ID NO: 15) or CCTCGTCCTGTTTGTC (SEQ ID NO: 31).

(13) The kit for evaluating a gene mutation according to (1), further comprising a wild type probe corresponding to a wild type of the JAK2, a wild type probe corresponding to a wild type of the CALR, and a wild type probe corresponding to a wild type of the MPL.

(14) The kit for evaluating a gene mutation according to (1), further comprising a primer set for amplifying a region comprising the gene mutation related to myeloproliferative neoplasms in JAK2, a primer set for amplifying a region comprising the gene mutation related to myeloproliferative neoplasms in CALR, and a primer set for amplifying a region comprising the a gene mutation related to myeloproliferative neoplasms in MPL.

(15) The kit for evaluating a gene mutation according to (1), comprising a microarray having the mutant probe immobilized on a carrier.

(16) The kit for evaluating a gene mutation according to (15), wherein the microarray has a wild type probe corresponding to a wild type of the JAK2, a wild type probe corresponding to a wild type of the CALR, and a wild type probe corresponding to a wild type of the MPL, each immobilized on the carrier.

(17) A data analysis method for a diagnosis of myeloproliferative neoplasms, comprising using the kit for evaluating a gene mutation according to any one of (1) to (16) to simultaneously identify a gene mutation related to myeloproliferative neoplasms in JAK2, a gene mutation related to myeloproliferative neoplasms in CALR and a gene mutation related to myeloproliferative neoplasms in MPL in a subject to be diagnosed.

(18) The data analysis method according to (17), wherein the kit for evaluating a gene mutation is a microarray having a mutant probe and a wild type probe for each of the gene mutations, and the data analysis method includes: measuring signals derived from the mutant probe and the wild type probe using the microarray; calculating a determination value 1 for each of the gene mutations by a formula: [mutant probe signal intensity]/([wild type probe signal intensity]+[mutant probe signal intensity]); and determining that the gene mutation is present when the calculated determination value 1 is higher than a predetermined cutoff value.

(19) The data analysis method according to (17), wherein the microarray has a wild type probe and a common probe that hybridizes with a region excluding the gene mutation related to myeloproliferative neoplasms in CALR, for a type 1 mutation, in which 52 bases from positions 513 to 564 are deleted in a nucleotide sequence represented by SEQ ID NO: 2, related to myeloproliferative neoplasms in CALR, and the data analysis method includes: measuring signals derived from the wild type probe and the common probe using the microarray; calculating a determination value 2 for the type 1 mutation by a formula: [wild type probe signal intensity]/[common probe signal intensity]; and determining that the gene mutation is absent when the calculated determination value 2 is higher than a predetermined cutoff value and determining that the gene mutation including the type 1 mutation is present when the calculated determination value 2 is lower than a predetermined cutoff value.

(20) The data analysis method according to (19), wherein the microarray further includes a mutant probe for the type 1 mutation, in which 52 bases from positions 513 to 564 are deleted in a nucleotide sequence represented by SEQ ID NO: 2, related to myeloproliferative neoplasms in CALR, and the data analysis method includes: measuring signals derived from the mutant probe, the wild type probe and the common probe using the microarray; calculating furthermore a different determination value for the type 1 mutation by a formula: [mutant probe signal intensity]/[common probe signal intensity]; and comparing the calculated different determination value with a predetermined cutoff value.

The present description encompasses the disclosure of Japanese Patent Application No. 2017-125903, which is the basis of the priority of the present application.

### Advantageous Effects of Invention

According to the present invention, it is possible to simultaneously identify the presence or absence of gene mutations present in especially JAK2, CALR and MPL among gene mutations related to myeloproliferative neoplasms. Thus, according to the present invention, the diagnostic accuracy of myeloproliferative neoplasms in a subject to be diagnosed utilizing the information of the gene mutations described above can be improved.

### BRIEF DESCRIPTION OF DRAWINGS

FIG. 1 schematically shows a type 1 mutation and a type 2 mutation related to myeloproliferative neoplasms in CALR.

FIG. 2 is a characteristic diagram in which the determination values are plotted for each gene mutation in the specimens used in the present Example 1.

FIG. 3 is a characteristic diagram in which the determination value 1 and the determination value 2 are plotted for the type 1 mutation of CALR in the specimens used in the present Example 1.

FIG. 4 is a characteristic diagram showing the relationship between the blocker concentration and determination value 1 in the hybridization experiment of the present Example 2.

FIG. 5 is a characteristic diagram showing the relationship between the mutation ratio (mutation %) and determination value 1 in the mutant sample of the hybridization experiment in the present Example 2.

### DESCRIPTION OF EMBODIMENTS

The kit for evaluating a gene mutation related to myeloproliferative neoplasms according to the present invention relates to gene mutations present in JAK2, CALR and MPL. These gene mutations present in JAK2, CALR and MPL are gene mutations employed for diagnosis of myeloproliferative neoplasms according to the classification by World Health Organization (WHO) (e.g., FY2016 version).

The kit for evaluating a gene mutation related to myeloproliferative neoplasms according to the present invention includes probe sets for identifying the gene mutations present in JAK2, CALR and MPL respectively.

Specifically, the gene mutation in JAK2 means a V617F mutation (substitution mutation at position 617 of valine to phenylalanine). This mutation contributes to the activation of JAK-STAT pathway and is a prominent feature in polycythemia vera (PV). In addition, the V617F mutation is observed at a frequency of 50 to 60% in patients with primary myelofibrosis (PMF) or patients with essential thrombocythemia (ET). The nucleotide sequence encoding a wild type of JAK2 is represented by SEQ ID NO: 1. In the case of the V617F mutation is present, G at position 351 in the nucleotide sequence represented by SEQ ID NO: 1 is mutated by substitution with T.

The gene mutation in CALR means a type 1 mutation of 52-base deletion and a type 2 mutation of 5-base insertion. The 52-base deletion and 5-base insertion are located at the C-terminus of the CALR protein. In patients with primary myelofibrosis (PMF) or patients with essential thrombocythemia (ET), either of these mutations is observed at a frequency of 20 to 25%. Mainly, type 2 mutation is associated with essential thrombocythemia (ET), and type 1 mutation is associated with primary myelofibrosis (PMF). The gene mutation in CALR is also a mutation found in myeloproliferative neoplasms in which the above-mentioned JAK2 gene mutation is not present. The nucleotide sequence encoding a wild type of CALR is represented by SEQ ID NO: 2. In the case of the type 1 mutation is present, 52 bases from positions 513 to 564 are deleted in a nucleotide sequence represented by SEQ ID NO: 2. In the case of the type 2 mutation is present, TTGTC is inserted between positions 568 and 569 in the nucleotide sequence represented by SEQ ID NO: 2.

Furthermore, the gene mutation in MPL means a W515K mutation (substitution mutation at position 515 of tryptophan to lysine) or a W515L mutation (substitution mutation at position 515 of tryptophan to leucine). The gene mutation in MPL is observed in patients with essential thrombocythemia (ET) at a frequency of 3 to 5% and in patients with primary myelofibrosis (PMF) at a frequency of 6 to 10%. The nucleotide sequence encoding a wild type of MPL is represented by SEQ ID NO: 3. In the case of the W515K mutation is present, TG at positions 305 and 306 in the nucleotide sequence represented by SEQ ID NO: 3 is mutated by substitution with AA. In the case of the W515L mutation is present, G at position 306 in the nucleotide sequence represented by SEQ ID NO: 3 is mutated by substitution with T.

More specifically, for the V617F mutation in JAK2, an oligonucleotide comprising, for example, CTCCACAGAaACATACTCC (SEQ ID NO: 4), corresponding to the substitution mutation in SEQ ID NO: 1 can be used as a mutant probe. In the sequence, the lowercase letter a corresponds to the substitution mutation at position 351 of G to T in the nucleotide sequence represented by SEQ ID NO: 1. In addition, when identifying the V617F mutation in JAK2, a wild type probe corresponding to a wild type of the JAK2 (a sequence in which the lowercase letter a in the above sequence is c) can also be used. In other words, in order to identify the V617F mutation in JAK2, a mutant probe comprising the nucleotide sequence represented by SEQ ID NO: 4 may be used, and a probe set including the mutant probe and the wild type probe may be used.

Furthermore, for the type 1 mutation in CALR, an oligonucleotide comprising, for example, TCCTTGT-CCTCTGCTCC (SEQ ID NO: 5), corresponding to the 52-base deletion in SEQ ID NO: 2 can be used as a probe. In the sequence, the position of hyphen “-” is a position of the 52-base deletion. In addition, when identifying the type 1 mutation in CALR, a wild type probe corresponding to a wild type of the CALR can also be used. In other words, in order to identify the type 1 mutation in CALR, a mutant probe comprising the nucleotide sequence represented by SEQ ID NO: 5 may be used, and a probe set including the mutant probe and the wild type probe may be used.

Furthermore, for the type 2 mutation in CALR, an oligonucleotide comprising, for example, ATCCTCCgacaaTTGTCCT (SEQ ID NO: 6) corresponding to the 5-base insertion in SEQ ID NO: 2 can be used as a probe. In the sequence, the lowercase letters gacaa are the 5-base insertion. In addition, when identifying the type 2 mutation in CALR, a wild type probe corresponding to a wild type of the CALR can also be used. In other words, in order to identify the type 2 mutation in CALR, a mutant probe comprising the nucleotide sequence represented by SEQ ID NO: 6 may be used, and a probe set including the mutant probe and the wild type probe may be used.

Furthermore, for the W515K mutation in MPL, an oligonucleotide comprising, for example, GAAACTGCttCCTCAGCA (SEQ ID NO: 7) corresponding to the substitution mutation in SEQ ID NO: 3 can be used as a mutant probe. In the sequence, the lowercase letters tt correspond to the substitution mutation of TG at positions 305 and 306 in the nucleotide sequence represented by SEQ ID NO: 3 with AA. In addition, when identifying the W515K mutation in MPL, a wild type probe corresponding to a wild type of the MPL (a sequence in which the lowercase letters tt in the above sequence is ca) can also be used. In other words, in order to identify the W515K mutation in MPL, a mutant probe comprising the nucleotide sequence represented by SEQ ID NO: 7 may be used, and a probe set including the mutant probe and the wild type probe may be used.

Furthermore, for the W515L mutation in MPL, an oligonucleotide comprising, for example, GGAAACTGCAaCCTCAG (SEQ ID NO: 8) corresponding to the substitution mutation in SEQ ID NO: 5 can be used as a mutant probe. In the sequence, the lowercase letter a corresponds to the substitution mutation of G at position 306 in the nucleotide sequence represented by SEQ ID NO: 3 with T. In addition, when identifying the W515L mutation in MPL, a wild type probe corresponding to a wild type of the MPL (a sequence in which the lowercase letter a in the above sequence is c) can also be used. In other words, in order to identify the W515L mutation in MPL, a mutant probe comprising the nucleotide sequence represented by SEQ ID NO: 8 may be used, and a probe set including the mutant probe and the wild type probe may be used.

Examples of each of the mutant probes for identifying gene mutations present in JAK2, CALR and MPL are shown as described above. However, the nucleotide sequences of the mutant probes are not limited to SEQ ID NOs: 4 to 8, and can be suitably designed based on the nucleotide sequence of JAK2 represented by SEQ ID NO: 1, the nucleotide sequence of CALR represented by SEQ ID NO: 2, and the nucleotide sequence of MPL represented by SEQ ID NO: 3.

The base length of these probes is not particularly limited, but can be, for example, 10 to 30 bases, and preferably 15 to 25 bases. The base length of the probes can be, for example, 10 to 30 bases, and preferably 15 to 25 bases as a total of base lengths of a nucleotide sequence designed based on a region comprising the gene mutation in the nucleotide sequence represented by SEQ ID NO: 1, 3, or 5 as described above and a nucleotide sequence added to one or both ends of the nucleotide sequence.

The probe designed as described above is preferably a nucleic acid, and more preferably a DNA. The DNA includes a double strand DNA and a single-stranded DNA, but is preferably a single-stranded DNA. The probe can be obtained, for example, by chemically synthesizing with a nucleic acid synthesizer. As the nucleic acid synthesizer, a device called DNA synthesizer, fully automatic nucleic acid synthesizer, automatic nucleic acid synthesizer or the like can be used.

The probe designed as described above is preferably used in the form of a microarray (for example, a DNA chip) by immobilizing the 5′ end of the probe on a carrier. The microarray preferably includes a mutant probe and a wild type probe for each of the gene mutations described above. By using a mutant probe and a wild type probe for each of the gene mutations, it is possible to accurately determine not only the presence or absence of the mutation but also the mutation rate. Here, the mutant probe and the wild type probe preferably have lengths with a difference of 2 bases or less, and more preferably have the same length.

The microarray according to the present invention can be manufactured by immobilizing the probes described above on a carrier.

The material for the carrier can be those known in the art and is not particularly limited. Examples of the material include noble metals such as platinum, platinum black, gold, palladium, rhodium, silver, mercury, tungsten, and compounds of these, and conductive materials such as carbon represented by graphite and carbon fiber; silicon materials represented by single crystal silicon, amorphous silicon, silicon carbide, silicon oxide and silicon nitride, and composite materials of these silicon materials represented by silicon on insulator (SOI) or the like; inorganic materials such as glass, quartz glass, alumina, sapphire, ceramics, forsterite, and photosensitive glass; organic materials such as polyethylene, ethylene, polypropylene, cyclic polyolefin, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene/acrylonitrile copolymer, acrylonitrile/butadiene styrene copolymer, polyphenylene oxide, and polysulfone. The shape of the carrier is also not particularly limited, but is preferably a flat plate shape.

In the present invention, a carrier having a carbon layer and a chemical modification group on the surface is preferably used as the carrier. The carrier having a carbon layer and a chemical modification group on the surface include one having a carbon layer and a chemical modification group on the surface of a substrate and one having a chemical modification group on the surface of a substrate composed of a carbon layer. The material for the substrate can be those known in the art, and is not particularly limited. The same material as those described as the material for the carrier can be used as the material for the substrate.

In a microarray according to the present invention, a carrier having a fine flat plate structure is preferably used. The shape is not limited, and examples thereof include a rectangle, square, or round shape. The carrier usually used has a shape of 1 to 75 mm square, preferably 1 to 10 mm square, more preferably 3 to 5 mm square. It is preferred to use a substrate made of a silicon material or a resin material, since they are easy to manufacture a carrier having a fine flat plate structure. In particular, it is more preferred that a carrier includes a substrate made of single crystal silicon, having on the surface a carbon layer and a chemical modification group. The single crystal silicon include one having slight changes in the orientation of the crystal axis in some parts (sometimes referred to as a mosaic crystal), or one having atomic scale disturbances (lattice defects).

The carbon layer formed on the substrate in the present invention is not particularly limited, but one made of synthetic diamond, high-pressure synthetic diamond, natural diamond, soft diamond (for example, diamond-like carbon), amorphous carbon, carbon-based material (for example, graphite, fullerene, carbon nanotube) or a mixture thereof, or one made of a laminate of these is preferably used. Furthermore, the carbon layer made of carbides such as hafnium carbide, niobium carbide, silicon carbide, tantalum carbide, thorium carbide, titanium carbide, uranium carbide, tungsten carbide, zirconium carbide, molybdenum carbide, chromium carbide and vanadium carbide may be used. Here, the soft diamond is a generic term for an incomplete diamond structure that is a mixture of diamond and carbon such as so-called diamond-like carbon (DLC), and the mixing ratio thereof is not particularly limited. The carbon layer is advantageous in that it is excellent in chemical stability and can withstand subsequent reactions in introducing chemical modification groups and binding to the analyte, in that its binding is flexible due to bonding with the analyte by electrostatic bonding, in that it is transparent to the detection system UV due to no UV absorption, and in that it can be energized during electroblotting. Furthermore, it is advantageous in that nonspecific adsorption is small in the binding reaction with the analyte. As described above, a carrier whose substrate itself is made of a carbon layer may be used.

In the present invention, the carbon layer can be formed by a known method. Examples of the method include a microwave plasma chemical vapor deposit (CVD) method, an electric cyclotron resonance chemical vapor deposit (ECRCVD) method, an inductive coupled plasma (ICP) method, a DC sputtering method, an electric cyclotron resonance (ECR) sputtering method, an ionization vapor deposition method, an arc vapor deposition method, a laser vapor deposition method, an electron beam (EB) vaporization method, and a resistance heating vaporization method.

In a high-frequency plasma CVD method, a raw material gas (methane) is decomposed by glow discharge generated between electrodes by a high frequency to synthesize a carbon layer on a substrate. In the ionization vapor deposition method, the raw material gas (benzene) is decomposed and ionized using thermoelectrons generated by a tungsten filament, and a carbon layer is formed on a substrate by a bias voltage. A carbon layer may be formed by the ionization vapor deposition method in a mixed gas composed of 1 to 99 vol. % hydrogen gas and 99 to 1 vol. % methane gas.

In the arc vapor deposition method, a DC voltage is applied between a solid graphite material (cathode evaporation source) and a vacuum vessel (anode) to cause arc discharge in vacuum to generate plasma of carbon atoms from the cathode, and by applying a bias voltage more negative than the evaporation source to a substrate, carbon ions in the plasma can be accelerated toward the substrate to form a carbon layer.

In the laser vapor deposition method, a carbon layer can be formed, for example, by irradiating a target plate of graphite with Nd:YAG laser (pulse oscillation) light to melt the same and depositing the carbon atoms on a glass substrate.

When forming a carbon layer on the surface of a substrate, the thickness of the carbon layer is usually about a single molecular layer to 100 μm. If the thickness is too small, the surface of the substrate of ground may be locally exposed, while if the thickness is too large, the productivity becomes poor. Thus, the thickness of the carbon layer is preferably from 2 nm to 1 μm, more preferably from 5 nm to 500 nm.

The oligonucleotide probe can be rigidly immobilized on the carrier by introducing a chemical modification group to the surface of a substrate in which the carbon layer is formed. The chemical modification group to be introduced can be appropriately selected by those skilled in the art and is not particularly limited, and examples thereof include an amino group, a carboxyl group, an epoxy group, a formyl group, a hydroxyl group, and an active ester group.

The introduction of an amino group can be performed, for example, by irradiating the carbon layer with ultraviolet light or subjecting the carbon layer to plasma treatment in ammonia gas. Alternatively, the introduction of an amino group can be performed by irradiating the carbon layer with ultraviolet light in chlorine gas to chlorinate, and further irradiating the chlorinated carbon layer with ultraviolet light in ammonia gas. Alternatively, the introduction of an amino group can be performed by reacting the chlorinated carbon layer with a polyvalent amine gas such as methylenediamine or ethylenediamine.

The introduction of a carboxyl group can be performed, for example, by reacting an appropriate compound with a carbon layer aminated as described above. Examples of the compound used for introducing a carboxyl group include halocarboxylic acids represented by a formula: X—R1-COOH (wherein X represents a halogen atom, and R1 represents a divalent hydrocarbon group having 10 to 12 carbon atoms) such as chloroacetic acid, fluoroacetic acid, bromoacetic acid, iodoacetic acid, 2-chloropropionic acid, 3-chloropropionic acid, 3-chloroacrylic acid, and 4-chlorobenzoic acid; divalent carboxylic acids represented by a formula: HOOC—R2-COOH (wherein R2 represents a single bond or a divalent hydrocarbon group having 1 to 12 carbon atoms) such as oxalic acid, malonic acid, succinic acid, maleic acid, fumaric acid, phthalic acid; polyvalent carboxylic acid such as polyacrylic acid, polymethacrylic acid, trimellitic acid, and butanetetracarboxylic acid; keto acids or aldehyde acids represented by a formula: R3-CO—R4-COOH (wherein R3 is a hydrogen atom or a divalent hydrocarbon group having 1 to 12 carbon atoms, and R4 represents a divalent hydrocarbon group having 1 to 12 carbon atoms); monohalides of dicarboxylic acid, represented by a formula: X—OC—R5-COOH (wherein X represents a halogen atom, and R5 represents a single bond or a divalent hydrocarbon group having 1 to 12 carbon atoms) such as succinic acid monochloride and malonic acid monochloride; and acid anhydrides such as phthalic anhydride, succinic anhydride, oxalic anhydride, maleic anhydride, and butanetetracarboxylic anhydride.

The introduction of an epoxy group can be performed, for example, by reacting an appropriate multivalent epoxy compound with a carbon layer aminated as described above. Alternatively, the introduction of an epoxy group can be performed by reacting an organic peracid with a carbon=carbon double bond contained in a carbon layer. Examples of the organic peracid include peracetic acid, perbenzoic acid, diperoxyphthalic acid, performic acid, and trifluoroperacetic acid.

The introduction of a formyl group can be performed, for example, by reacting glutaraldehyde with a carbon layer aminated as described above.

The introduction of a hydroxyl group can be performed, for example, by reacting water with a carbon layer chlorinated as described above.

The active ester group refer to a group of ester which has a high acidity electron attractive group on the alcohol side of the ester group to activate a nucleophilic reaction, that is, an ester group with high reactive activity. The active ester group is an ester group having an electron attractive group on the alcohol side of the ester group and more activated than alkyl ester. The active ester group has reactivity with groups such as an amino group, a thiol group, and a hydroxyl group. More specifically, phenol esters, thiophenol esters, N-hydroxyamine esters, cyanomethyl esters, and esters of heterocyclic hydroxy compounds are known as the active ester groups having much higher activity than alkyl ester or the like. More specifically, examples of the active ester group include a p-nitrophenyl group, an N-hydroxysuccinimide group, a succinimide group, a phthalimide group and a 5-norbornene-2,3-dicarboximide group. In particular, an N-hydroxysuccinimide group is preferably used as the active ester group.

The introduction of the active ester group can be performed, for example, by causing active esterification to the carboxyl group introduced as described above with a dehydrating condensing agent such as cyanamide or carbodiimide (for example, 1-[3-(dimethylamino)propyl]-3-ethylcarbodiimide) and a compound such as N-hydroxysuccinimide. By this active esterification process, a group in which an active ester group such as an N-hydroxysuccinimide group is bonded to the end of a hydrocarbon group via an amide bond can be formed (JP Patent Publication (Kokai) No. 2001-139532 A).

The microarray having the probe immobilized on a carrier can be produced by dissolving the probe in a spotting buffer to prepare a spotting solution, dispensing the prepared solution into a 96- or 384-well plastic plate, and spotting the dispensed solution on the carrier with a spotter device or the like. Alternatively, the spotting solution may be spotted manually with a micropipette.

After the spotting, it is preferred to perform an incubation in order to promote the reaction in which the probe binds to the carrier. The incubation is performed usually at a temperature of −20 to 100° C., preferably 0 to 90° C., and usually for 0.5 to 16 hours, preferably for 1 to 2 hours. It is preferred that the incubation is performed under a high humidity condition, for example, a condition of 50 to 90% humidity. Following the incubation, it is preferred to perform washing using a washing solution (for example, 50 mM TBS/0.05% Tween20, 2×SSC/0.2% SDS solution, ultrapure water, or the like) to remove DNA not bound to the carrier.

By using the microarray configured as described above, it is possible to simultaneously determining the presence or absence of gene mutations for the gene mutations present in JAK2, CALR and MPL respectively in a subject to be diagnosed.

Specifically, determination of the presence or absence of the gene mutations present in JAK2, CALR and MPL includes a step of extracting DNA from a sample derived from a subject to be diagnosed, and a step of amplifying a region comprising the gene mutation in JAK2, a region comprising the gene mutation in CALR and a region comprising the gene mutation in MPL respectively using the extracted DNA as a template, and a step of detecting the presence or absence of the gene mutations present in JAK2, CALR and MPL respectively, which are included in the amplified nucleic acids, using the microarray described above.

The subject to be diagnosed is generally a human, and there is no particular limitation on the race etc. In particular, the subject to be diagnosed is the yellow race, preferably the East Asian race, and particularly preferably Japanese. Further, the subject to be diagnosed can be a patient suspected of having a myeloproliferative neoplasm.

The sample derived from the subject to be diagnosed is not particularly limited. Examples thereof include blood-related samples (blood, serum, and plasma), lymph fluid, feces, cancer cells, tissue, and crushed and extracted organs.

First, DNA is extracted from a sample collected from the subject to be diagnosed. The extraction means is not particularly limited. For example, a DNA extraction method using phenol/chloroform, ethanol, sodium hydroxide, or CTAB, can be used.

Next, an amplification reaction is performed using the obtained DNA as a template to amplify a region containing JAK2, a region containing CALR, and a region containing MPL. As the amplification reaction, polymerase chain reaction (PCR), loop-mediated isothermal amplification (LAMP), isothermal and chimeric primer-initiated amplification of nucleic acids (ICAN), and like methods can be applied. In the amplification reaction, it is desirable to add a label so as to be able to identify the amplified region. In this case, the method for labeling the amplified nucleic acid is not particularly limited. For example, a method of previously labeling a primer used in the amplification reaction may be used, or a method using a labeled nucleotide as a substrate in the amplification reaction may be used. The labeling substance is not particularly limited, and radioisotopes, fluorescent dyes, or organic compounds, such as digoxigenin (DIG) and biotin, can be used.

Moreover, this reaction system contains a buffer necessary for nucleic acid amplification and labeling, a heat-resistant DNA polymerase, a primer specific to the amplified region, labeled nucleotide triphosphate (specifically, nucleotide triphosphate with a fluorescent label, etc.), nucleotide triphosphate, and magnesium chloride.

The primer used in the amplification reaction of a region comprising the above gene mutation in JAK2 is not particularly limited, as long as it can specifically amplify the region comprising the gene mutation. Such a primer can be appropriately designed by a person skilled in the art. For example, a primer set can be used, comprising:

(SEQ ID NO: 9)
primer JAK2-F: 5′-GAGCAAGCTTTCTCACAAGCATTTGG-3′;
and
(SEQ ID NO: 10)
primer JAK2-R: 5′-CTGACACCTAGCTGTGATCCTGAAACTG-3′.

The primer used in the amplification reaction of a region comprising the above gene mutation in CALR is not particularly limited, as long as it can specifically amplify the region comprising the gene mutation. Such a primer can be appropriately designed by a person skilled in the art. For example, a primer set can be used, comprising:

(SEQ ID NO: 11)
primer CALR-F: 5′-CGTAACAAAGGTGAGGCCTGGT-3′;
and
(SEQ ID NO: 12)
primer CALR-R: 5′--GGCCTCTCTACAGCTCGTCCTTG-3′.

The primer used in the amplification reaction of a region comprising the above gene mutation in MPL is not particularly limited, as long as it can specifically amplify the region comprising the gene mutation. Such a primer can be appropriately designed by a person skilled in the art. For example, a primer set can be used, comprising:

(SEQ ID NO: 13)
primer MPL-F: 5′-CTCCTAGCCTGGATCTCCTTGG-3′;
and
(SEQ ID NO: 14)
primer MPL-R: 5′--ACAGAGCGAACCAAGAATGCCTGTTTAC-3′.

The nucleic acid fragment amplified by a primer is not particularly limited, as long as it contains a region corresponding to the designed probe. For example, a fragment of 1 kbp or less is preferable, a fragment of 800 bp or less is more preferable, a fragment of 500 bp or less is even more preferable, and a fragment of 350 bp or less is particularly preferable.

A hybridization reaction is performed between the amplified nucleic acid obtained as described above and the probe immobilized on the carrier to detect hybridization of the amplified nucleic acid with the mutant probe, whereby the presence or absence of the above gene mutation in the subject to be diagnosed can be evaluated. That is, hybridization of the amplified nucleic acid with the mutant probe can be measured, for example, by detecting a label.

Regarding signals from labels, for example, when a fluorescent label is used, a fluorescence signal is detected by a fluorescence scanner and analyzed by image analysis software to thereby quantify the signal intensity. The hybridization reaction is preferably carried out under stringent conditions. The stringent conditions refer to conditions where specific hybrids are formed and non-specific hybrids are not formed. For example, the stringent conditions refer to conditions where a hybridization reaction is carried out at 50° C. for 16 hours, followed by washing with 2×SSC/0.2% SDS at 25° C. for 10 minutes, and with 2×SSC at 25° C. for 5 minutes. Alternatively, the hybridization temperature can be set to 45 to 60° C. when the salt concentration is 0.5×SSC. When the chain length of the probe is short, the hybridization temperature is more preferably lower than the above range; and when the chain length is long, the hybridization temperature is more preferably higher than the above range. It goes without saying that the specific hybridization temperature increases as the salt concentration increases, whereas the specific hybridization temperature decreases as the salt concentration decreases.

Moreover, when a microarray including a mutant probe and a wild type probe is used for each of the gene mutations described above, the presence or absence of the gene mutation can be evaluated using the signal intensities from the mutant probe and wild type probe. Specifically, the signal intensity in the wild type probe and the signal intensity in the mutant probe are each measured, and a determination value for evaluating the signal intensity derived from the mutant probe is calculated. As an example of calculating the determination value, for example, there is a method using formula: [mutant probe-derived signal intensity]/([wild type probe-derived signal intensity]+[mutant probe-derived signal intensity]).

Then, the determination value calculated by the above formula is compared with a predetermined threshold value (cutoff value). When the determination value is higher than the threshold value, it is determined that the amplified nucleic acid contains the above gene mutation. When the determination value is lower than the threshold value, it is determined that the amplified nucleic acid does not contain the above gene mutation. Use of the determination value in this way makes it possible to determine the presence or absence of each of the gene mutations in JAK2, CALR, and MPL.

Here, the threshold value is not particularly limited, but can be specified, for example, based on a determination value calculated by the above formula using a specimen in which each of the gene mutations present in JAK2, CALR, and MPL is determined to be wild type. More specifically, a plurality of determination values is calculated using a plurality of specimens in which each of the gene mutations present in JAK2, CALR, and MPL is determined to be wild type, and their average value +3σ (σ: standard deviation) can be used as the threshold value. Average value +2σ or average value +σ can also be used as the threshold value.

It is known that, in addition to type 1 of 52-base deletion, the gene mutations present in CALR described above include, for example, 46-base deletion mutation, 34-base deletion mutation, 24-base deletion mutation, and other mutations similar to type 1 (these are collectively referred to as type 1-like mutations) in type 1 mutation sites; and that, in addition to the type 1 mutation, these type 1-like mutations are also involved in diseases (Leukemia (2016) 30, 431-438). Leukemia (2016) 30, 431-438 indicates that in portions where type 1 of 52-base deletion and type 1-like mutations occur, there are other mutations that are not classified into these mutations. Therefore, if the presence or absence of the above-mentioned type 1 gene mutation, type 1-like gene mutations, and other mutations that are not classified into these mutations can be detected as the gene mutations present in CALR, it will be useful information for the definitive diagnosis of MPN.

Use of the determination value calculated by the above formula makes it possible to determine type 1 of 52-base deletion, separately from the type 1-like mutations and other mutations mentioned above. That is, when the determination value is higher than the threshold value, it can be determined that type 1 of 52-base deletion is present. When the determination value is lower than the threshold value, it can be determined that the amplified nucleic acid is a wild type with no mutation, or contains any of the type 1-like mutations and other mutations.

The presence or absence of the gene mutations present in CALR described above may be determined using a determination value calculated by the above formula; alternatively, the presence or absence thereof may be determined using a determination value calculated by a different formula. A determination value calculated by the above formula is referred to as “determination value 1,” and a determination value calculated by a formula different from the above formula is referred to as “determination value 2.” That is, the presence or absence of the gene mutations present in CALR described above may be determined using the “determination value 1” or the “determination value 2,” or both of the “determination value 1” and the “determination value 2.”

Specifically, use of the determination value 2 specified below makes it possible to accurately determine whether there is no mutation, and whether there is any of type 1 of 52-base deletion, the type 1-like mutations, and other mutations that are not classified into these mutations. The determination value 2 is a value obtained by dividing the signal intensity derived from the wild type probe by the signal intensity derived from a common probe, which is different from the mutant probe and the wild type probe. The common probe is a nucleotide comprising a nucleotide sequence complementary to a region common to a wild-type amplified nucleic acid and a mutant amplified nucleic acid, for the type 1 gene mutation. That is, the common probe specifically hybridizes with the amplified nucleic acid, regardless of the presence or absence of the type 1 gene mutation contained in the amplified nucleic acid.

The common probe is not particularly limited, and can be an oligonucleotide comprising a sequence of positions 397 to 659 in a nucleotide sequence of CALR gene represented by SEQ ID NO: 2. More specific examples of the common probe include an oligonucleotide comprising CTCCTCATCCTCATCTTTGTC (SEQ ID NO: 15). Other examples of the common probe include an oligonucleotide comprising CCTCCTCATCCTCATCTTTGTC (SEQ ID NO: 26) and an oligonucleotide comprising CCTCCTTGTCCTCCTCAT (SEQ ID NO: 27). Still other examples of the common probe include an oligonucleotide comprising CCTCGTCCTGTTTGTCC (SEQ ID NO: 31).

Specifically, the formula for calculating the determination value 2 can be [wild type probe-derived signal intensity]/[common probe-derived signal intensity]. The determination value 2 calculated by formula: [wild type probe-derived signal intensity]/[common probe-derived signal intensity] decreases when a mutation (deletion or insertion) is present in a position corresponding to the wild type probe in the amplified nucleic acid. When the determination value 2 is lower than the threshold value, it is determined that the amplified nucleic acid contains any of type 1 of 52-base deletion, the type 1-like mutations, and other mutations that are not classified into these mutations. When the obtained determination value 2 is higher than the threshold value, it is determined that the amplified nucleic acid does not contain any of type 1 of 52-base deletion, the type 1-like mutations, and other mutations that are not classified into these mutations. Use of the determination value 2 in this way makes it possible to identify that there is any of the above-mentioned CALR type 1 gene mutation, type 1-like gene mutations, and other mutations, separately from amplified nucleic acids that do not contain any of these mutations.

In contrast, as a determination value different from the determination values 1 and 2 described above, a determination value calculated by formula: [mutant probe-derived signal intensity]/[common probe-derived signal intensity] may be used to identify the type 1 gene mutation. Specifically, the determination value calculated by this formula is a value that increases when the amplified nucleic acid contains the type 1 gene mutation. When the determination value is higher than the threshold value, it can be determined that the amplified nucleic acid contains type 1 of 52-base deletion.

Moreover, the above “determination value 1” and “determination value 2” may be used together to determine the presence or absence of the gene mutations present in CALR described above. Use of the “determination value 1” and the “determination value 2” makes it possible to accurately determine whether there is the type 1 mutation of 52-base deletion, or any of the type 1-like mutations and other mutations, or none of these mutations. Specifically, when the determination value 2 is lower than the threshold value, and the determination value 1 is higher than the threshold value, it is determined that the amplified nucleic acid contains the type 1 gene mutation. When the determination value 2 is lower than the threshold value, and the obtained determination value 1 is lower than the threshold value, it is determined that the amplified nucleic acid contains any of the type 1-like gene mutations and other mutations. When the determination value 2 is higher than the threshold value, and the determination value 1 is lower than the threshold value, it is determined that the amplified nucleic acid does not contain any of the type 1, type 1-like, and other mutations. Use of the determination values 1 and 2 in this way makes it possible to identify the above-mentioned CALR type 1 gene mutation, separately from the type 1-like and other mutations in type 1 mutation sites.

In contrast, when the type 2 mutation is determined among the gene mutations present in CALR described above, only the “determination value 1” may be used, or the “determination value 2” may be used, or the “determination value 1” and the “determination value 2” may be used together, as in the case of the type 1 mutation. As for the type 2 gene mutation described above, there are also mutations similar to type 2 (type 2-like mutations) (Leukemia (2016) 30, 431-438). It is indicated that in portions where type 2 and type 2-like mutations occur, there are other mutations that are not classified into these mutations. Therefore, use of the “determination value 2” makes it possible to identify that there is any of the type 2 gene mutation, type 2-like gene mutations, and other mutations, separately from amplified nucleic acids that do not contain any of these mutations. Further, use of the “determination value 1” and the “determination value 2” together makes it possible to identify the type 2 gene mutation, separately from the type 2-like and other mutations.

As described above, each of the gene mutations present in JAK2, CALR, and MPL can be identified simultaneously by using a microarray including a mutant probe for identifying each of the gene mutations present in JAK2, CALR, and MPL. In particular, each gene mutation can be identified with high accuracy by using the determination value 1, or using the determination value 1 and the determination value 2 together. The information on the gene mutations present in JAK2, CALR, and MPL can be used, for example, for the diagnosis of myeloproliferative neoplasms in the WHO Classification (2016 version). Specifically, according to the WHO Classification, the presence of the above gene mutation in JAK2 is one requirement for the diagnosis of polycythemia vera (PV). Moreover, according to the WHO Classification, the presence of any of the gene mutations present in JAK2, CALR, and MPL is one requirement for the diagnosis of essential thrombocythemia (ET). Furthermore, according to the WHO Classification, the presence of any of the gene mutations present in JAK2, CALR, and MPL is one requirement for the diagnosis of prefibrotic/early primary myelofibrosis (prefibrotic/early PMF) or primary myelofibrosis (PMF).

Thus, a microarray including a mutant probe for identifying each of the gene mutations present in JAK2, CALR, and MPL can be used, for example, for the diagnosis of myeloproliferative neoplasms based on the WHO Classification (2016 version).

### EXAMPLES

Hereinafter, the present invention will be further described in detail by examples, but the technical scope of the present invention is not limited thereto.

### Example 1

### 1. Sample Preparation

In the present example, peripheral blood (patient specimen from which written informed consent was obtained), collected by clinical research using Yamaguchi University Hospital as a main facility, was used as specimens. Sample DNA was extracted from these specimens as follows. Peripheral blood leukocyte genomic DNA was extracted by a conventional method (NaI method).

Using the DNA sample prepared as described above, predetermined regions of the JAK2 gene, the CALR gene and the MPL gene were each amplified by PCR. The primer set shown in Table 1 was designed for this PCR. In the primer set shown in Table 1, a fluorescent label (IC5) is added to the forward primer with “F”.

TABLE 1

| Primer | | |
|---|---|---|
| name | Sequence (5′-3′) | SEQ ID NO |
| JAK2-F | GAGCAAGCTTTCTCACAAGCATTTGG | SEQ ID NO: 9 |
| JAK2-R | CTGACACCTAGCTGTGATCCTGAAACTG | SEQ ID NO: 10 |
| CALR-F | CGTAACAAAGGTGAGGCCTGGT | SEQ ID NO: 11 |
| CALR-R | GGCCTCTCTACAGCTCGTCCTTG | SEQ ID NO: 12 |
| MPL-F | CTCCTAGCCTGGATCTCCTTGG | SEQ ID NO: 13 |
| MPL-R | ACAGAGCGAACCAAGAATGCCTGTTTAC | SEQ ID NO: 14 |

A primer mix was prepared by mixing the primer sets designed as described above to have the composition shown in Table 2.

TABLE 2

| Reagent name | Concentration after mixing (μM) |
|---|---|
| TE buffer | |
| JAK2-F | 2.0 |
| JAK2-R | 0.7 |
| CALR-F | 1.2 |
| CALR-R | 0.4 |
| MPL-F | 4.0 |
| MPL-R | 1.3 |

A PCR reaction solution having the composition shown in Table 3 was prepared using the DNA sample and primer mix prepared as described above.

TABLE 3

| Reagent name | Manufacturer | Content (μL) |
|---|---|---|
| 10x PCR Buffer | Roche Diagnostics | 2.0 |
| 10 mM dNTP mix | Roche Diagnostics | 0.4 |
| Faststart DNA taq polymerase | Roche Diagnostics | 0.2 |
| Primer mix | Life Technologies Japan | 2.0 |
| DNA sample (2 ng/μL) | | 5.0 |
| Purified water | | 10.4 |

Then, after 5 minutes at 95° C., 40 PCR thermal cycles were performed, with 30 seconds at 95° C., 30 seconds at 59° C. and 45 seconds at 72° C. as 1 cycle, followed by 10 minutes at 72° C., and finally maintaining at 4° C.

### 2. Microarray

In the present example, a mutant probe corresponding to the V617F mutation in the JAK2 gene, the type 1 mutation and type 2 mutation in the CALR gene, and the W515L/K mutation in the MPL gene, and a wild type probe corresponding thereto were designed.

In addition, in the present example, a common probe, which corresponds to a site that is commonly present in the region amplified by the above primer for the CALR gene, regardless of whether it has a type 1 mutation or not, was designed. That is, the common probe specifically hybridizes with the amplified nucleic acids regardless of the presence or absence of a type 1 gene mutation contained in the amplified nucleic acid.

The base sequences of the designed probes are shown in Table 4.

TABLE 4

| Probe name | Sequence | SEQ ID NO |
|---|---|---|
| JAK2 V617F wild type | TTTTTTTTTTTTCTCCACAGACACATACTCC | SEQ ID NO: 16 |
| JAK2 V617F mutant | TTTTTTTTTTTTCTCCACAGAAACATACTCC | SEQ ID NO: 17 |
| MPL W515 wild type | TTTTTTTTTTTTAAACTGCCACCTCAGC | SEQ ID NO: 18 |
| MPL W515L mutant | TTTTTTTTTTTTGGAAACTGCAACCTCAG | SEQ ID NO: 19 |
| MPL W515K mutant | TTTTTTTTTTTTGAAACTGCTTCCTCAGCA | SEQ ID NO: 20 |
| CALR type1 - wild type | TTTTTTTTTTTTCTCTTTGCGTTTCTTGTCTTCT | SEQ ID NO: 21 |
| CALR type1 - mutant | TTTTTTTTTTTTTCCTTGTCCTCTGCTCC | SEQ ID NO: 22 |
| CALR type2- wild type | TTTTTTTTTTTTCCTCCTTGTCCTCTGC | SEQ ID NO: 23 |
| CALR type2- mutant | TTTTTTTTTTTTATCCTCCGACAATTGTCCT | SEQ ID NO: 24 |
| Common probe | TTTTTTTTTTTTCCTCGTCCTGTTTGTCC | SEQ ID NO: 25 |

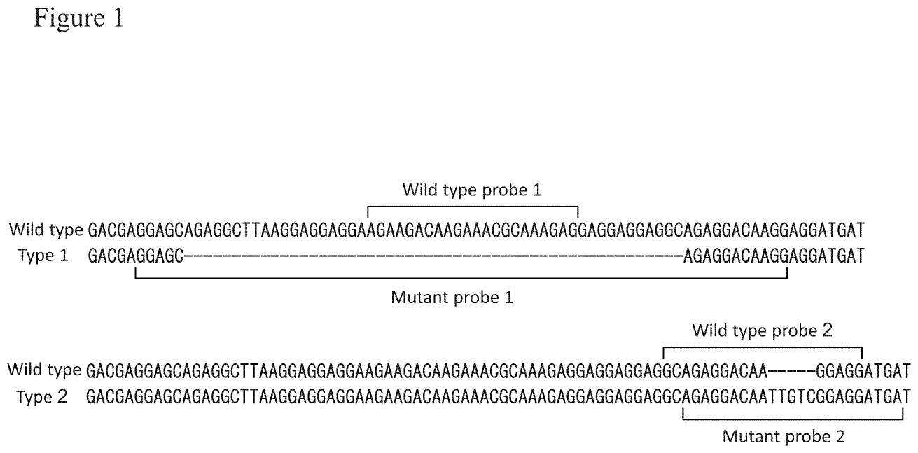

For type 1 and type 2 gene mutations in CALR, a wild type probe 1 and a mutant probe 1, and a wild type probe 2 and a mutant probe 2 were designed, respectively, as shown in FIG. 1. In FIG. 1, the 52-base deletion, which is a type 1 gene mutation in CALR, is indicated by “-”. Moreover, in FIG. 1, for the 5-base insertion, which is a type 2 gene mutation in CALR, the corresponding wild type region is indicated by “-”.

### 3. Identification of Gene Mutation

Hybridization was performed as follows using a chip having the above probes. First, a humidity box was placed in a chamber set at a specified temperature (52° C.), and the chamber and humidity box were sufficiently preheated. 4 μL of the PCR reaction solution was mixed with 2 μL of hybridization buffer (2.25×SSC/0.23% SDS/0.2 nM Cy5-labeled oligo DNA (manufactured by Sigma-Aldrich)), 3 μL of this solution was collected and added dropwise on the central projecting part of a Hybricover, this was put on the chip, and reacted for 1 hour in a hybridization chamber apparatus (manufactured by Toyo Kohan) set at 52° C. After the hybridization reaction, a cleaning stainless steel holder was immersed in a 0.1×SSC/0.1% SDS solution, and the chip with the Hybricover removed was set on the holder. After shaking up and down several times, the holder was immersed in a 1×SSC solution (room temperature) until detecting the fluorescence intensity of the chip.

Immediately before detection, the chip was covered with a cover film, and the fluorescence intensity of the chip was detected with BIOSHOT (manufactured by Toyo Kohan). Using the fluorescence intensities of the wild type probe and the mutant probe measured as described above, a determination value 1 was calculated for the gene mutations of JAK2, the gene mutations of CALR and the gene mutations of MPL by the following formula. Determination value 1=[fluorescence intensity of mutant probe]/([fluorescence intensity of wild type probe]+[fluorescence intensity of mutant probe])

For the type 1 gene mutation of CALR, a determination value 2 was calculated by the following formula.

Determination value 2=[fluorescence intensity of wild type probe]/[fluorescence intensity of common probe]

In the present example, determination value 1 was calculated using specimens in which the gene mutations of JAK2, the gene mutations of CALR and the gene mutations of MPL were all wild type (n=4), and the average value and standard deviation of determination value 1 were determined. Then, the average value +3σ or the average value +4σ (σ: standard deviation) was set as the cutoff value (see table below).

TABLE 5

| | JAK2 | MPL | MPL | CALR | CALR |
|---|---|---|---|---|---|
| | V617F | W515L | W515K | type1 | type2 |
| Ave | 0.165 | 0.091 | 0.013 | 0.183 | 0.061 |
| Σ | 0.028 | 0.018 | 0.005 | 0.036 | 0.016 |
| Cutoff | 0.249 | 0.145 | 0.028 | 0.290 | 0.110 |
| | Ave +3σ | Ave +4σ | Ave +4σ | Ave +3σ | Ave +4σ |

Furthermore, in the present example, the cutoff value was defined for determination value 2 as follows. That is, determination value 2 was calculated using specimens that were confirmed to have no mutation at the type 1 mutation site (n=40), and the average value and standard deviation of determination value 2 were determined. Then, the average value −1.5σ was set as the cutoff value (see table below).

TABLE 6

| | |
|---|---|
| Ave | 1.480 |
| σ | 0.202 |
| Cutoff | 1.177 |
| | Ave −1.5σ |

### 4. Results

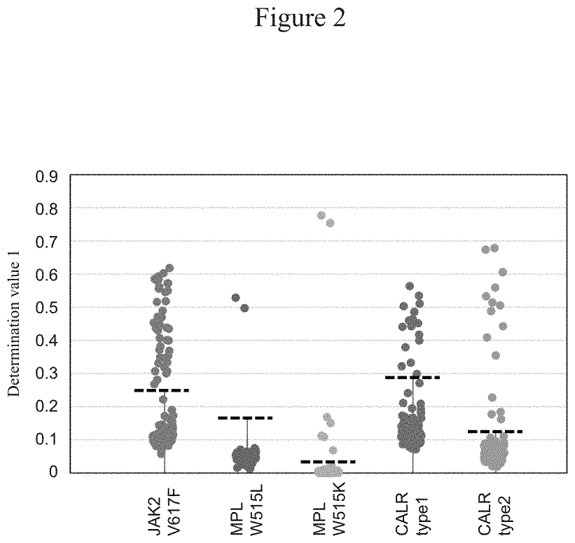

The results of plotting for each gene mutation the determination value 1 calculated as described above for each specimen used in the present example, are shown in FIG. 2. In FIG. 2, the cutoff value defined as described above is indicated by a dashed line. The plot below the dashed lines indicates the specimens that are wild-type (not having a gene mutation) for each gene mutation, and the plot above the dashed lines indicates the specimens that have each gene mutation.

As shown in FIG. 2, it was found that the mutant and wild type could be identified with high accuracy by the determination value 1 for the gene mutation of JAK2, the type 2 gene mutation of CALR, and the gene mutations of MPL. However, as shown in FIG. 2, for the type 1 gene mutation of CALR, there were many specimens that were plotted in the vicinity of the cutoff value, and the determination accuracy may have been low for the type 1 gene mutation with only the determination value 1.

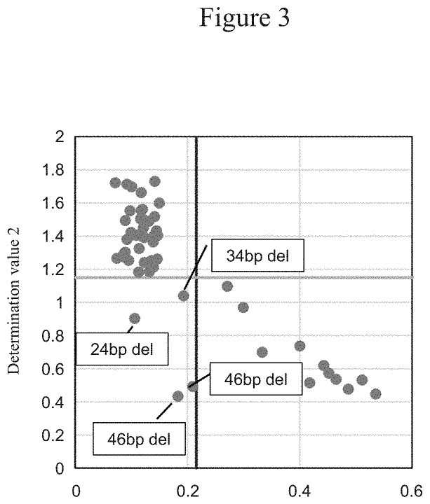

Therefore, in the present example, determination value 1 and determination value 2 were used for the determination of the type 1 gene mutation of CALR. Specifically, for each specimen used in the present example, the determination value 1 and the determination value 2 calculated for CALR type 1 were plotted on a graph with the horizontal axis as determination value 1 and the vertical axis as determination value 2, as shown in FIG. 3. As shown in FIG. 3, the plot of each specimen was divided into four regions partitioned by the cutoff value defined for determination value 1 and the cutoff value defined for determination value 2. When the gene mutations were determined for each plotted specimen by other methods, the specimens that exceeded the cutoff value defined for determination value 1 and that were below the cutoff value defined for determination value 2 had all a type 1 gene mutation of CALR, that is, a 52-base deletion. Meanwhile, it was found that the specimens that were below the cutoff value defined for determination value 1 and that were below the cutoff value defined for determination value 2 were all 46-base deletions, 34-base deletions or 24-base deletions, which are similar to a type 1 gene mutation of CALR.

The results of the present example shows that type 1-like mutant genes such as 46-base deletion, 34-base deletion and 24-base deletion, which could not be distinguished from the wild type by determination value 1 alone, can be identified distinctively from the wild type by using determination value 2.

### Example 2

In the present example, it was verified whether the detection sensitivity of the gene mutations of the JAK2 gene, the type 1 mutation of the CALR gene and the gene mutations of the MPL gene is improved by designing a blocker that specifically hybridizes with the amplified product derived from the wild type for each of the JAK2 gene, CALR gene, and MPL gene to suppress that the amplified product derived from the wild type non-specifically hybridizes with the mutant probe. The type 2 mutation of the CALR gene is different from the wild type by 5 bases, and non-specific hybridization is unlikely to occur in the present example, therefore a blocker was not designed.

### 1. Sample Adjustment

In the present example, genomic DNA derived from healthy human peripheral blood leukocytes (purchased from Biochain) diluted to 8 ng/μL with TE buffer was used as a wild type sample.

Moreover, in the present example, a mutant sample was prepared as follows. In the present example, wild type plasmids and mutant plasmids were made through FASMAC's Artificial Gene Synthesis service. As a wild type plasmid for the JAK2 gene, a plasmid into which a region consisting of 400 bases at positions 151 to 550 in the base sequence shown in SEQ ID NO: 1 was inserted, was used. As a wild type plasmid for the CALR gene, a plasmid into which a region consisting of 389 bases at positions 376 to 764 in the base sequence shown in SEQ ID NO: 2 was inserted, was used. As a wild type plasmid for the MPL gene, a plasmid into which a region consisting of 399 bases at positions 107 to 505 in the base sequence shown in SEQ ID NO: 3 was inserted, was used. As the mutant plasmid for each gene, a plasmid into which the same region was inserted except that it had the mutations described above (V617F mutation of JAK2 gene, W515L mutation or W515K mutation of MPL gene and type 1 mutation or type 2 mutation of CALR gene), was used. The purchased plasmid DNA was used dissolved in TE buffer so as to be about 100 ng/μL and further diluted to about 1 ng/μL with TE buffer.

Then, three kinds of plasmids, one corresponding to the wild type of the JAK2 gene, one corresponding to the wild type of the MPL gene and one corresponding to the wild type of the CALR gene, were mixed and diluted with TE buffer to prepare a wild type plasmid mix with the concentration of each plasmid at about 200 pg/μL. In addition, three kinds of plasmids, one corresponding to the V617F mutant of the JAK2 gene, one corresponding to the W515L mutant of the MPL gene and one corresponding to the type 1 mutant of the CALR gene, were mixed and diluted with TE buffer to prepare a 100% mutant plasmid mix A with the concentration of each plasmid at about 200 pg/μL. Similarly, three kinds of plasmids, one corresponding to the V617F mutant of the JAK2 gene, one corresponding to the W515K mutant of the MPL gene and one corresponding to the type 2 mutant of the CALR gene, were mixed and diluted with TE buffer to prepare a 100% mutant plasmid mix B with the concentration of each plasmid at about 200 pg/μL.

In the present example, a mutant sample was prepared by mixing these wild type plasmid mix and 100% mutant plasmid mix A or 100% mutant plasmid mix B at a predetermined ratio. After the preparation of the mutant sample, the mutation % for the JAK2 gene and the MPL gene (the ratio of mutants to the total of wild types and mutants) was quantified by digital PCR, and the mutation % for the CALR gene was quantified by fragment analysis. Then, the mutant sample was diluted to about 0.16 pg/μL and used for PCR.

In the present example, using the wild type sample and mutant sample prepared as described above, predetermined regions of the JAK2 gene, CALR gene and MPL gene were each amplified by PCR under the same conditions as in Example 1.

In the present example, using the microarray used in Example 1, a hybridization buffer was made in the same manner as in Example 1 except that a blocker was included, to perform a hybridization experiment. In the present example, a hybridization buffer was prepared so that the JAK2 gene blocker, CALR gene blocker, and MPL gene blocker shown in Table 7 each had a concentration of 90 to 210 nM. Then, the PCR reaction solution and the hybridization buffer were mixed at a ratio of 2:1 and the hybridization experiment was performed.

TABLE 7

| Blocker | Base sequence (5′→3′) | SEQ ID NO |
|---|---|---|
| JAK2 gene blocker | CTCCACAGACACATACTCC | 28 |
| MPL gene blocker | AAACTGCCACCTCAGC | 29 |
| CALR type1 gene | CCTCCTCCTCTTTGCG | 30 |
| blocker | | |

In the present example, the determination value 1 was calculated as a result of the hybridization experiment in the same manner as in Example 1. However, in the present example, unlike Example 1, for the detection of the W515L mutant of the MPL gene, the formula: determination value 1=[fluorescence intensity of W515L mutant probe]/([fluorescence intensity of wild type probe]+[fluorescence intensity of W515L mutant probe]+[fluorescence intensity of W515K mutant probe]) was followed, and for the detection of the W515K mutant of the MPL gene, the formula: determination value 1=[fluorescence intensity of W515K mutant probe]/([fluorescence intensity of wild type probe]+[fluorescence intensity of W515L mutant probe]+[fluorescence intensity of W515K mutant probe]) was followed.

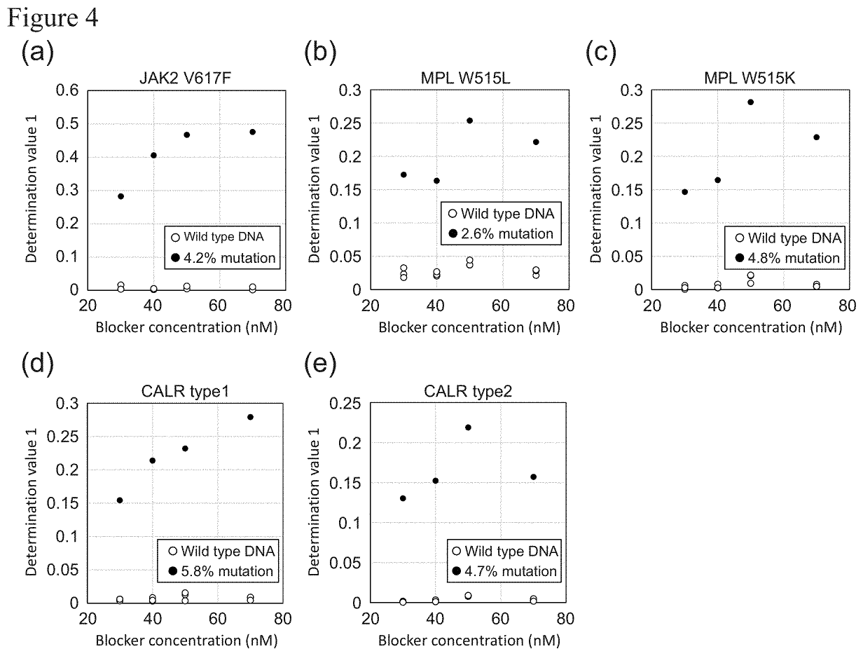

The relationship between the blocker concentration and the determination value 1 is shown in FIG. 4. FIG. 4 (a) shows the results when the V617F mutant of the JAK2 gene was detected, (b) shows the results when the W515L mutant of the MPL gene was detected, (c) shows the results when the W515K mutant of the MPL gene was detected, (d) shows the results when the type 1 mutant of the CARL gene was detected, and (e) shows the results when the type 2 mutant of the CARL gene was detected. As shown in FIG. 4, it was found that by adding a blocker to the hybridization buffer, the mutant form of a gene can be detected with excellent detection sensitivity even if the mutation ratio is 2.6 to 5.8%.

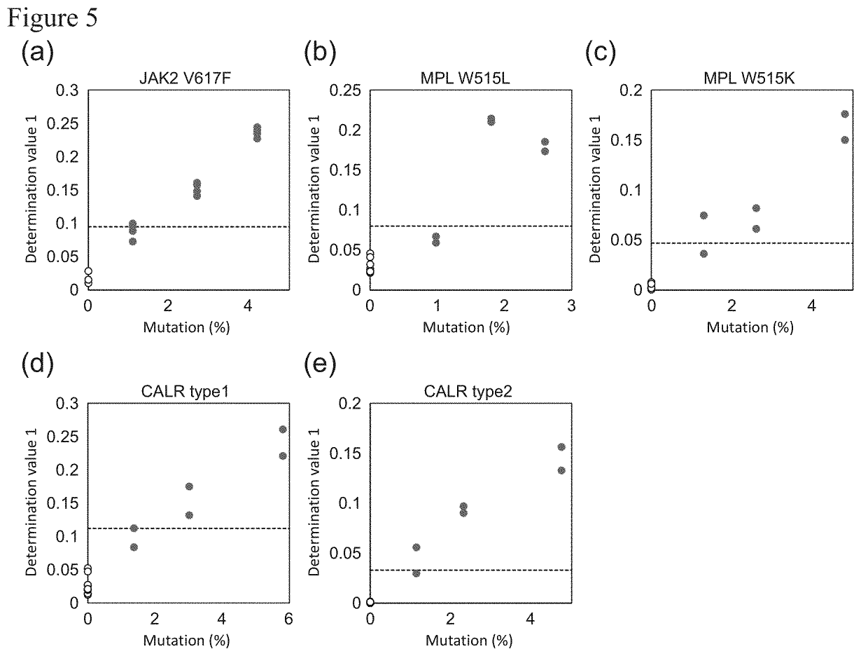

Moreover, in the present example, hybridization experiments were similarly performed by adjusting the ratio of mutant forms of each gene by changing the mixing ratio of the wild type plasmid mix and the mutant plasmid mix A or B in the mutant sample. The relationship between the mutation ratio (mutation %) in the mutant sample and determination value 1 is shown in FIG. 5. FIG. 5 (a) shows the results when the V617F mutant of the JAK2 gene was detected, (b) shows the results when the W515L mutant of the MPL gene was detected, (c) shows the results when the W515K mutant of the MPL gene was detected, (d) shows the results when the type 1 mutant of the CARL gene was detected, and (e) shows the results when the type 2 mutant of the CARL gene was detected. As shown in FIG. 5, it was found that by adding a blocker to the hybridization buffer, excellent detection sensitivity can be achieved even if the mutation ratio in the mutant sample is about 2%. In the hybridization experiment of which the results are shown in FIG. 5, the JAK2 gene blocker concentration was 150 nM, the CALR gene blocker concentration was 210 nM, and the MPL gene blocker concentration was 150 nM.

The results of the measurement of the actual concentrations of each mutant with respect to the theoretical values of the mutation ratio contained in the mutant sample are shown in Table 8. The measured values shown in Table 8 are the results of the quantification of the mutation % for the JAK2 gene and the MPL gene by digital PCR, and of the quantification of the mutation % for the CALR gene by fragment analysis.

TABLE 8

| | Measured value (%) | | | | |
|---|---|---|---|---|---|
| Theoretical | JAK2 | MPL | CALR | MPL | CALR |
| value (%) | V617F | W515L | type1 | W515K | type2 |
| 10 | 11.5 | 7.9 | 11.2 | 12.2 | 10.6 |
| 5 | 4.2 | 2.6 | 5.8 | 4.8 | 4.7 |
| 2.5 | 2.7 | 1.8 | 3.0 | 2.6 | 2.3 |
| 1.25 | 1.1 | 0.98 | 1.4 | 1.3 | 1.1 |

All publications, patents and patent applications cited in the present specification are hereby incorporated by reference in their entirety.

1. A kit for evaluating a gene mutation related to myeloproliferative neoplasms, comprising:
a mutant probe that specifically hybridizes with a gene mutation related to myeloproliferative neoplasms in JAK2,
a mutant probe that specifically hybridizes with a gene mutation related to myeloproliferative neoplasms in CALR, and
a mutant probe that specifically hybridizes with a gene mutation related to myeloproliferative neoplasms in MPL.

2. The kit for evaluating a gene mutation according to claim 1, wherein the gene mutation related to myeloproliferative neoplasms in JAK2 is a V617F mutation.

3. The kit for evaluating a gene mutation according to claim 1, wherein the gene mutation related to myeloproliferative neoplasms in CALR is a type 1 mutation of 52-base deletion in which 52 bases from positions 513 to 564 are deleted in a nucleotide sequence represented by SEQ ID NO: 2 and/or a type 2 mutation of 5-base insertion in which TTGTC is inserted between positions 568 and 569 in the nucleotide sequence represented by SEQ ID NO: 2.

4. The kit for evaluating a gene mutation according to claim 1, wherein the gene mutation related to myeloproliferative neoplasms in MPL is a W515K mutation and/or W515L mutation.

5. The kit for evaluating a gene mutation according to claim 1, comprising a common probe that hybridizes with a region excluding the gene mutation related to myeloproliferative neoplasms in CALR.

6. The kit for evaluating a gene mutation according to claim 1, wherein the mutant probe that specifically hybridizes with a gene mutation related to myeloproliferative neoplasms in JAK2 is an oligonucleotide comprising CTCCACAGAAACATACTCC (SEQ ID NO: 4).

7. The kit for evaluating a gene mutation according to claim 1, wherein the gene mutation related to myeloproliferative neoplasms in CALR is a type 1 mutation of 52-base deletion in which 52 bases from positions 513 to 564 are deleted in a nucleotide sequence represented by SEQ ID NO: 2, and the mutant probe that specifically hybridizes with the gene mutation related to myeloproliferative neoplasms in CALR is an oligonucleotide comprising TCCTTGTCCTCTGCTCC (SEQ ID NO: 5).

8. The kit for evaluating a gene mutation according to claim 1, wherein the gene mutation related to myeloproliferative neoplasms in CALR is a type 2 mutation of 5-base insertion in which TTGTC is inserted between positions 568 and 569 in the nucleotide sequence represented by SEQ ID NO: 2, and the mutant probe that specifically hybridizes with the gene mutation related to myeloproliferative neoplasms in CALR is an oligonucleotide comprising ATCCTCCGACAATTGTCCT (SEQ ID NO: 6).

9. The kit for evaluating a gene mutation according to claim 1, wherein the gene mutation related to myeloproliferative neoplasms in MPL is a W515K mutation, and the mutant probe that specifically hybridizes with the gene mutation related to myeloproliferative neoplasms in MPL is an oligonucleotide comprising GAAACTGCTTCCTCAGCA (SEQ ID NO: 7).

10. The kit for evaluating a gene mutation according to claim 1, wherein the gene mutation related to myeloproliferative neoplasms in MPL is a W515L mutation, and the mutant probe that specifically hybridizes with the gene mutation related to myeloproliferative neoplasms in MPL is an oligonucleotide comprising GGAAACTGCAACCTCAG (SEQ ID NO: 8).

11. The kit for evaluating a gene mutation according to claim 5, wherein the common probe is an oligonucleotide comprising a sequence of positions 397 to 659 in a nucleotide sequence of CALR gene represented by SEQ ID NO: 2.

12. The kit for evaluating a gene mutation according to claim 5, wherein the common probe is an oligonucleotide comprising CTCCTCATCCTCATCTTTGTC (SEQ ID NO: 15) or CCTCGTCCTGTTTGTC (SEQ ID NO: 31).

13. The kit for evaluating a gene mutation according to claim 1, further comprising a wild type probe corresponding to a wild type of the JAK2, a wild type probe corresponding to a wild type of the CALR, and a wild type probe corresponding to a wild type of the MPL.

14. The kit for evaluating a gene mutation according to claim 1, further comprising a primer set for amplifying a region comprising the gene mutation related to myeloproliferative neoplasms in JAK2, a primer set for amplifying a region comprising the gene mutation related to myeloproliferative neoplasms in CALR, and a primer set for amplifying a region comprising the gene mutation related to myeloproliferative neoplasms in MPL.

15. The kit for evaluating a gene mutation according to claim 1, comprising a microarray having the mutant probe immobilized on a carrier.

16. The kit for evaluating a gene mutation according to claim 15, wherein the microarray has a wild type probe corresponding to a wild type of the JAK2, a wild type probe corresponding to a wild type of the CALR, and a wild type probe corresponding to a wild type of the MPL, each immobilized on the carrier.

17. A data analysis method for diagnosis of myeloproliferative neoplasms, comprising using a kit for evaluating a gene mutation related to myeloproliferative neoplasms, wherein the kit comprises a mutant probe that specifically hybridizes with a gene mutation related to myeloproliferative neoplasms in JAK2, a mutant probe that specifically hybridizes with a gene mutation related to myeloproliferative neoplasms in CALR, and a mutant probe that specifically hybridizes with a gene mutation related to myeloproliferative neoplasms in MPL, to simultaneously identify the gene mutation related to myeloproliferative neoplasms in JAK2, a gene mutation related to myeloproliferative neoplasms in CALR and the gene mutation related to myeloproliferative neoplasms in MPL in a subject to be diagnosed,
the kit for evaluating a gene mutation comprising a microarray having a mutant probe and a wild type probe for each of the gene mutations,
the microarray having a mutant probe, a wild type probe and a common probe that hybridizes with a region excluding the gene mutation related to myeloproliferative neoplasms in CALR, for a type 1 mutation, in which 52 bases from positions 513 to 564 are deleted in a nucleotide sequence represented by SEQ ID NO: 2, related to myeloproliferative neoplasms in CALR and/or a type 2 mutation of 5-base insertion in which TTGTC is inserted between positions 568 and 569 in the nucleotide sequence represented by SEQ ID NO: 2,
the data analysis method comprising:
measuring signals derived from the mutant probe, the wild type probe and the common probe using the microarray;
calculating a determination value 1 for the type 1 mutation and/or the type 2 mutation by a formula: [mutant probe signal intensity]/([wild type probe signal intensity]+[mutant probe signal intensity]);
calculating a determination value 2 for the type 1 mutation and/or the type 2 mutation by a formula: [wild type probe signal intensity]/[common probe signal intensity];
determining that the type 1 mutation and/or the type 2 mutation is present when the calculated determination value 1 is higher than a predetermined cutoff value and the calculated determination value 2 is lower than a predetermined cutoff value;
determining that a gene mutation similar to the type 1 mutation and/or a gene mutation similar to the type 2 mutation is present when the calculated determination value 1 is lower than a predetermined cutoff value and the calculated determination value 2 is lower than a predetermined cutoff value; and
determining that none of the type 1 mutation, the gene mutation similar to the type 1 mutation, the type 2 mutation, and the gene mutation similar to the type 2 mutation are present when the calculated determination value 1 is lower than a predetermined cutoff value and the calculated determination value 2 is higher than a predetermined cutoff value.

18. (canceled)

19. The data analysis method according to claim 17, wherein the microarray has a wild type probe and a common probe that hybridizes with a region excluding the gene mutation related to myeloproliferative neoplasms in CALR, for a type 1 mutation, in which 52 bases from positions 513 to 564 are deleted in a nucleotide sequence represented by SEQ ID NO: 2, related to myeloproliferative neoplasms in CALR, and the data analysis method comprises: measuring signals derived from the wild type probe and the common probe using the microarray; calculating a determination value 2 for the type 1 mutation by a formula: [wild type probe signal intensity]/[common probe signal intensity]; and determining that the gene mutation is absent when the calculated determination value 2 is higher than a predetermined cutoff value and determining that the gene mutation including the type 1 mutation is present when the calculated determination value 2 is lower than a predetermined cutoff value.

20. (canceled)

21. The data analysis method according to claim 17, wherein the gene mutation related to myeloproliferative neoplasms in CALR is a type 1 mutation of 52-base deletion in which 52 bases from positions 513 to 564 are deleted in a nucleotide sequence represented by SEQ ID NO: 2, and the mutant probe that specifically hybridizes with the gene mutation related to myeloproliferative neoplasms in CALR is an oligonucleotide comprising TCCTTGTCCTCTGCTCC (SEQ ID NO: 5).

22. The data analysis method according to claim 17, wherein the gene mutation related to myeloproliferative neoplasms in CALR is a type 2 mutation of 5-base insertion in which TTGTC is inserted between positions 568 and 569 in the nucleotide sequence represented by SEQ ID NO: 2, and the mutant probe that specifically hybridizes with the gene mutation related to myeloproliferative neoplasms in CALR is an oligonucleotide comprising ATCCTCCGACAATTGTCCT (SEQ ID NO: 6).

23. The data analysis method according to claim 17, wherein the common probe is an oligonucleotide comprising a sequence of positions 397 to 659 in a nucleotide sequence of CALR gene represented by SEQ ID NO: 2.

24. The data analysis method according to claim 17, wherein the common probe is an oligonucleotide comprising CTCCTCATCCTCATCTTTGTC (SEQ ID NO: 15) or CCTCGTCCTGTTTGTC (SEQ ID NO: 31).

25. The data analysis method according to claim 17, wherein the gene mutation related to myeloproliferative neoplasms in JAK2 is a V617F mutation.

26. The data analysis method according to claim 17, wherein the gene mutation related to myeloproliferative neoplasms in MPL is a W515K mutation and/or W515L mutation.

27. The data analysis method according to claim 17, wherein the mutant probe that specifically hybridizes with a gene mutation related to myeloproliferative neoplasms in JAK2 is an oligonucleotide comprising CTCCACAGAAACATACTCC (SEQ ID NO: 4).

28. The data analysis method according to claim 17, wherein the gene mutation related to myeloproliferative neoplasms in MPL is a W515K mutation, and the mutant probe that specifically hybridizes with the gene mutation related to myeloproliferative neoplasms in MPL is an oligonucleotide comprising GAAACTGCTTCCTCAGCA (SEQ ID NO: 7).

29. The data analysis method according to claim 17, wherein the gene mutation related to myeloproliferative neoplasms in MPL is a W515L mutation, and the mutant probe that specifically hybridizes with the gene mutation related to myeloproliferative neoplasms in MPL is an oligonucleotide comprising GGAAACTGCAACCTCAG (SEQ ID NO: 8).

30. The data analysis method according to claim 17, wherein the kit for evaluating a gene mutation further comprises a primer set that amplifies a region comprising the gene mutation related to myeloproliferative neoplasms in JAK2, a primer set that amplifies a region comprising the gene mutation related to myeloproliferative neoplasms in CALR, and a primer set that amplifies a region comprising the a gene mutation related to myeloproliferative neoplasms in MPL.

31. A kit for evaluating a gene mutation related to myeloproliferative neoplasms, the kit being used for the data analysis method according to claim 17,
the kit comprising:
a microarray having a mutant probe that specifically hybridizes with a gene mutation related to myeloproliferative neoplasms in JAK2, a mutant probe that specifically hybridizes with a gene mutation related to myeloproliferative neoplasms in CALR, and a mutant probe that specifically hybridizes with a gene mutation related to myeloproliferative neoplasms in MPL, wild type probes corresponding to each wild type of the gene mutations, and a common probe that hybridizes with a region excluding the gene mutation related to myeloproliferative neoplasms in CALR, each immobilized on a carrier;
a primer set that amplifies a region comprising the gene mutation related to myeloproliferative neoplasms in JAK2;
a primer set that amplifies a region comprising the gene mutation related to myeloproliferative neoplasms in CALR; and
a primer set that amplifies a region comprising the gene mutation related to myeloproliferative neoplasms in MPL.


SEQUENCE LISTING (31 sequences)

SEQ ID NO: 1 - DNA, 700 bp, Homo sapiens
tatgggtcaagcctgtttgactggcattattcatgattcctgtaccactcttgctctctctcactttgatctccatattccaggcttacacaggggtttcctcagaacgttgatggcagttgcaggtccatataaagggaccaaagcacattgtatcctcatctatagtcatgctgaaagtaggagaaagtgcatctttattatggcagagagaattttctgaactatttatggacaacagtcaaacaacaattctttgtacttttttttttccttagtctttctttgaagcagcaagtatgatgagcaagctttctcacaagcatttggttttaaattatggagtatgtgtctgtggagacgagagtaagtaaaactacaggctttctaatgcctttctcagagcatctgtttttgtttatatagaaaattcagtttcaggatcacagctaggtgtcagtgtaaactataatttaacaggagttaagtatttttgaaactgaaaacactgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaactta

SEQ ID NO: 2 - DNA, 1062 bp, Homo sapiens
ggctgattttttgtatttttagtagagacggggtttcaccgtgttagccagggtggtctcgatctcctgacctcgtgatccgcccaccttggcctctgggcgaggattacaggcgtgatccacctcacctggcctctccatctttttaactgcagtgtcagcggtgttccttgtcttctctgcagatgcaggcagcagaatatagtggttataggaacacaggtggaaaccctgtccaaagcaagggctatcgggtatcacctctgaccatccttcccattcatcctccaggtcaagtctggcaccatctttgacaacttcctcatcaccaacgatgaggcatacgctgaggagtttggcaacgagacgtggggcgtaacaaaggtgaggcctggtcctggtcctgatgtcgggggcgggcagggctggcagggggcaaggccctgaggtgtgtgctctgcctgcaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagg

SEQ ID NO: 3 - DNA, 611 bp, Homo sapiens
cacagcgcctgcgccagggactgggcgccgggtgcgagtggggcggggctcggagaggggcgaggcgcggggcgcggagaggggcggggccctgaccttgcgggccgacggctgcgcaggtgcccgcagtgcccaggggcggcgaggggcggggccagagtaggggctggctggatgagggcggggctccggcccgggtgggccgaagtctgaccctttttgtctcctagcctggatctccttggtgaccgctctgcatctagtgctgggcctcagcgccgtcctgggcctgctgctgctgaggtggcagtttcctgcacactacaggtaccgcccccgccaggcaggagactggcggtggaccaggtggagccgaaggcctgtaaacaggcattcttggttcgctctgtgaccccagatctccgtccaccgcccgtgcgcacctacggcttcgccacttcctgcacgtcacctctgggactcgccgcggctccttacactctaacacgcccactataccgcccacctcgaacagccccgcctcctgctgctcacctcggcgactaggccaccgtccacccttcagccaaactgcccactccacccccatc

SEQ ID NO: 4 - DNA, 19 bp, Artificial
  Synthetic DNA
ctccacagaaacatactcc

SEQ ID NO: 5 - DNA, 17 bp, Artificial
  Synthetic DNA
tccttgtcctctgctcc

SEQ ID NO: 6 - DNA, 19 bp, Artificial
  Synthetic DNA
atcctccgacaattgtcct

SEQ ID NO: 7 - DNA, 18 bp, Artificial
  Synthetic DNA
gaaactgcttcctcagca

SEQ ID NO: 8 - DNA, 17 bp, Artificial
  Synthetic DNA
ggaaactgcaacctcag

SEQ ID NO: 9 - DNA, 26 bp, Artificial
  Synthetic DNA
gagcaagctttctcacaagcatttgg

SEQ ID NO: 10 - DNA, 28 bp, Artificial
  Synthetic DNA
ctgacacctagctgtgatcctgaaactg

SEQ ID NO: 11 - DNA, 22 bp, Artificial
  Synthetic DNA
cgtaacaaaggtgaggcctggt

SEQ ID NO: 12 - DNA, 23 bp, Artificial
  Synthetic DNA
ggcctctctacagctcgtccttg

SEQ ID NO: 13 - DNA, 22 bp, Artificial
  Synthetic DNA
ctcctagcctggatctccttgg

SEQ ID NO: 14 - DNA, 28 bp, Artificial
  Synthetic DNA
acagagcgaaccaagaatgcctgtttac

SEQ ID NO: 15 - DNA, 21 bp, Artificial
  Synthetic DNA
ctcctcatcctcatctttgtc

SEQ ID NO: 16 - DNA, 31 bp, Artificial
  Synthetic DNA
ttttttttttttctccacagacacatactcc

SEQ ID NO: 17 - DNA, 31 bp, Artificial
  Synthetic DNA
ttttttttttttctccacagaaacatactcc

SEQ ID NO: 18 - DNA, 28 bp, Artificial
  Synthetic DNA
ttttttttttttaaactgccacctcagc

SEQ ID NO: 19 - DNA, 29 bp, Artificial
  Synthetic DNA
ttttttttttttggaaactgcaacctcag

SEQ ID NO: 20 - DNA, 30 bp, Artificial
  Synthetic DNA
ttttttttttttgaaactgcttcctcagca

SEQ ID NO: 21 - DNA, 34 bp, Artificial
  Synthetic DNA
ttttttttttttctctttgcgtttcttgtcttct

SEQ ID NO: 22 - DNA, 29 bp, Artificial
  Synthetic DNA
tttttttttttttccttgtcctctgctcc

SEQ ID NO: 23 - DNA, 28 bp, Artificial
  Synthetic DNA
ttttttttttttcctccttgtcctctgc

SEQ ID NO: 24 - DNA, 31 bp, Artificial
  Synthetic DNA
ttttttttttttatcctccgacaattgtcct

SEQ ID NO: 25 - DNA, 29 bp, Artificial
  Synthetic DNA
ttttttttttttcctcgtcctgtttgtcc

SEQ ID NO: 26 - DNA, 22 bp, Artificial
  Synthetic DNA
cctcctcatcctcatctttgtc

SEQ ID NO: 27 - DNA, 18 bp, Artificial
  Synthetic DNA
cctccttgtcctcctcat

SEQ ID NO: 28 - DNA, 19 bp, Artificial
  Synthetic DNA
ctccacagacacatactcc

SEQ ID NO: 29 - DNA, 16 bp, Artificial
  Synthetic DNA
aaactgccacctcagc

SEQ ID NO: 30 - DNA, 16 bp, Artificial
  Synthetic DNA
cctcctcctctttgcg

SEQ ID NO: 31 - DNA, 17 bp, Artificial
  Synthetic DNA
cctcgtcctgtttgtcc